(19) United States
(12) Patent Application Publication
(10) Pub. No.: US 2011/0311970 A1
(43) Pub. Date: Dec. 22, 2011

(54) COMPOSITIONS AND METHODS FOR INTRACELLULAR ANALYTE ANALYSIS

(76) Inventors: Catherine M. SHACHAF, Palo Alto, CA (US); Garry P. NOLAN, Palo Alto, CA (US)

(21) Appl. No.: 13/087,056

(22) Filed: Apr. 14, 2011

Related U.S. Application Data
(60) Provisional application No. 61/342,534, filed on Apr. 14, 2010.

(51) Int. Cl. C12Q 1/68; G01N 33/566

(52) U.S. Cl. 435/6.11; 435/6.19; 435/7.23; 435/7.24

(57) ABSTRACT

Compositions and methods for multiplex immunodetection of analytes in single cells or cell populations are described. The invention utilizes analytical nanotags (ANTs) each specific for a different target analyte (TA) species. Analytical nanotags typically comprise biocompatible composite organic-inorganic nanoparticles (bCOINs) that include probe molecules specific for a particular TA species. A plurality of ANTs each specific for a different TA species can be used in a single multiplex assay, including assays for analyzing intracellular analytes in living cells.

### RELATED APPLICATION

This application claims the benefit of and priority to commonly owned U.S. provisional patent application Ser. No. 61/342,534, filed 14 Apr. 2010, which is herein incorporated by reference in its entirety for any and all purposes.

### FEDERALLY-SPONSORED RESEARCH OR DEVELOPMENT

This invention was made with U.S. government support under National Cancer Institute grant number NCI U54 RFA-CA-05-024. As such the U.S. government may have certain rights in this invention.

### BACKGROUND OF THE INVENTION

1. Field of the Invention

The invention relates generally to the analysis of specific analyte species in complex environments, such as in cells and tissues in vitro or in vivo, in environmental samples, etc. More particularly, the invention concerns compositions and methods for multiplex analysis of samples known or suspected to contain one or more different target analyte species.

The following description includes information that may be useful in understanding the present invention. It is not an admission that any of the information provided herein, or any publication specifically or implicitly referenced herein, is prior art, or even particularly relevant, to the presently claimed invention.

2. Background

To better understand the complex processes occurring in abnormal cells compared to normal cells, there is an urgent need to improve the technology for simultaneous detection of multiple events in a single cell. Multiplex, or parallel, reaction processes can increase efficiencies of biochemical or clinical analyses; however, when using such reactions it becomes important to develop a probe identification system that has distinguishable components for each probe species in a large probe set (i.e., a mixture containing a plurality of different probe species).

When coupled with surface marker definitions of cell type, intracellular analysis for particular target analyte species can be a powerful tool for understanding the biochemistry of primary cell samples. To date, antibodies labeled with fluorescent molecules have been most commonly used for this purpose. However, one rapidly reaches limits on the numbers of simultaneous measurements that can be taken based on conventional fluorophore detection approaches. The use of up to 17 different fluorescent molecules has been reported. As is well understood, however, the often overlapping spectra of fluorophores requires compensation and becomes more difficult to carry out with each additional probe. Therefore, there is a need to develop molecules that overcome the limitations of fluorescence in multi-color detection schemes.

The instant invention addresses these needs, as described below, particularly in the context of multiplex analyzes of intracellular analytes.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. See, e.g., IMMUNOBIOLOGY, Fifth Edition, C. A. Janeway, P. Travers, M., Walport, M. J. Shlomchiked., ed. Garland Publishing (2001). The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments, and binding agents that employ the complementarity determining regions (CDRs) of the parent antibody, or variants thereof that retain antigen binding activity. Antibodies are defined herein as retaining at least one desired activity of the parent antibody. Desired activities can include the ability to bind the antigen specifically, the ability to inhibit proleration in vitro, the ability to inhibit angiogenesis in vivo, and the ability to alter cytokine profile(s) in vitro.

Native antibodies (native immunoglobulins) are usually heterotetrameric glycoproteins of about 150,000 Daltons, typically composed of two identical light (L) chains and two identical heavy (H) chains. The heavy chain is approximately 50 kD in size, and the light chain is approximately 25 kDa. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known

An “antibody derivative” is an immune-derived moiety, i.e., a molecule that is derived from an antibody. This includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding and include Fab, Fab′, F(ab′)₂, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994).

An “antibody variant” refers herein to a molecule which differs in amino acid sequence from the amino acid sequence of a native or parent antibody that is directed to the same antigen by virtue of addition, deletion and/or substitution of one or more amino acid residue(s) in the antibody sequence and which retains at least one desired activity of the parent anti-binding antibody. Desired activities can include the ability to bind the antigen specifically, the ability to inhibit proliferation in vitro, the ability to inhibit angiogenesis in vivo, and the ability to alter cytokine profile in vitro. The amino acid change(s) in an antibody variant may be within a variable region or a constant region of a light chain and/or a heavy chain, including in the Fc region, the Fab region, the CH₁ domain, the CH₂ domain, the CH₃ domain, and the hinge region.

The term “biologically active,” in the context of an antibody or antibody fragment or variant, refers to an antibody or antibody fragment or antibody variant that is capable of binding the desired epitope under physiological or assay conditions.

A “biomolecule” is a specific biochemical in a cell that has a particular molecular feature or role that makes it of interest.

An “epitope” or “antigenic determinant” refers to that portion of an antigen that reacts with an antibody antigen-binding portion derived from an antibody.

The word “label” when used herein refers to a detectable compound or composition, such as one that is conjugated directly or indirectly to a target-specific probe molecule. The label may itself be detectable by itself (e.g., a Raman label, a radioisotope, a fluorescent label, etc.) or, in the case of an enzymatic label, may catalyze chemical alteration of a substrate compound or composition that is detectable.

A “ligand” is a biomolecule that is able to bind to and form a complex with a biomolecule to serve a biological purpose. Thus an antigen may be described as a ligand of the antibody to which it binds.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The term “multispecific antibody” can refer to an antibody, or a monoclonal antibody, having binding properties for at least two different epitopes. In one embodiment, the epitopes are from the same antigen. In another embodiment, the epitopes are from two or more different antigens. Methods for making multispecific antibodies are known in the art. Multispecific antibodies include bispecific antibodies (having binding properties for two epitopes), trispecific antibodies (three epitopes) and so on.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

A “plurality” means more than one.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

The term “species” is used herein in various contexts, e.g., a particular species of chemotherapeutic agent. In each context, the term refers to a population of chemically indistinct molecules of the sort referred in the particular context.

The term “specific” or “specificity” in the context of probe-target analyte refers to the selective, non-random interaction between a probe molecule and its target analyte. For example, in the context of antibody-antigen interactions the “antigen” refers to a molecule that is recognized and bound by an antibody molecule or other immune-derived moiety. This interaction depends on the presence of structural, hydrophobic/hydrophilic, and/or electrostatic features that allow appropriate chemical or molecular interactions between the molecules. Thus, an antibody (or other probe class) is commonly said to “bind” (or “specifically bind”) or be “reactive with” (or “specifically reactive with), or, equivalently, “reactive against” (or “specifically reactive against”) its target analyte antigen. Antibodies are commonly described in the art as being “against” or “to” their antigens as shorthand for antibody binding to the antigen. Antibody and other probe molecules can be tested for specificity of binding by comparing binding to the desired target analyte to binding to unrelated analytes or analyte analogues antigen under a given set of conditions. Preferably, a probe according to the invention will lack significant binding to molecules other than the target analyte, or even analogs of the target analyte.

“Specifically associate”, “specific association”, “specific binding”, “specific hybridization” and the like refer to a specific, non-random interaction between two molecules (for example, a probe molecule and its target analyte), which interaction depends on the presence of structural, hydrophobic/hydrophilic, and/or electrostatic features that allow appropriate chemical or molecular interactions between the molecules.

Herein, “stable” refers to an interaction between two molecules (e.g., a probe and a target analyte molecule) that is sufficiently stable such that the molecules can be maintained for the desired purpose or manipulation. For example, a “stable” interaction between a probe and its target refers to one wherein the probe becomes and remains associated with a target for a period sufficient to achieve the desired effect or to make the desired measurement or other analysis.

### SUMMARY OF THE INVENTION

One aspect of the invention concerns methods for intracellular analyte analysis. Such methods comprise contacting a population of cells known or suspected to contain or present a target analyte (TA) species with an analytical nanotag (ANT) species comprising a TA species-specific probe bound to a biocompatible composite organic-inorganic nanoparticle (bCOIN) and then analyzing the cells using any suitable process (e.g., Raman spectroscopy) to assess intracellular formation of TA:ANT complexes, thereby conducting an intracellular analyte analysis. In some embodiments the cells are living or metabolically active, while in other embodiments the cells are not living, and in some cases are preserved, preferably in a fixative.

Preferred target analytes include protein (e.g., enzymes, receptors, transcription factors, etc.), a peptides, small molecules (e.g., second messengers, drugs, substrates, etc.), and nucleic acids (e.g., chromosomal or mitochondrial DNA, RNA molecules, including small nuclear RNAs, ribsosomal RNAs, transfer RNAs, interfering RNAs, etc.).

Preferably, the analytical nanotag (ANT) species comprises a biocompatible COIN (bCOIN) species comprised of a metal species, optionally copper, gold, palladium, platinum, or silver, and an entrapped organic Raman label species. In certain embodiments, Raman label species are active organic compounds are polycyclic aromatic or heteroaromatic compounds. Typically a Raman label compound has a molecular weight less than about 500 Daltons. Exemplary Raman-active organic compounds include, but are not limited to, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo-(3,4-d)pyrimidine, 8-mercaptoadenine, 9-amino-acridine, and the like. Additional representative examples of Raman-active organic compounds include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins, aminoacridine, and the like. These and other Raman-active organic compounds may be obtained from commercial sources (e.g., Molecular Probes, Eugene, Oreg.).

In other preferred embodiments an analytical nanotag (ANT) species comprises clustered COIN species that include a metal, optionally copper, gold, palladium, platinum, or silver, and an entrapped organic Raman label species, wherein the clustered COIN species is coated with a biocompatible layer conjugated to the TA species-specific probe. Preferred TA species-specific probes include antibodies and antibody fragments, receptors and receptor fragments, receptor ligands, nucleic acid molecules (e.g., synthetic oligonucleotides), enzyme substrates, drug molecules and the their metabolites, etc.

In some embodiments, a biocompatible COIN is protein-encapsulated COIN, for example, with an albumin species, examples of which include bovine and human serum albumin.

In certain embodiments the instant methods are configured for simultaneous analysis of a plurality of TA species, wherein the population of cells is contacted with a plurality of ANT species each comprising a different TA species-specific probe species and different biocompatible COIN species.

These and other aspects and embodiments of the invention are described in greater detail in the following detailed description, accompanying figures, and the claims.

### BRIEF DESCRIPTION OF THE FIGURES

This application contains at least one figure executed in color. Copies of this application with color drawing(s) will be provided upon request and payment of the necessary fee. A brief summary of each of the figures is provided below.

FIG. 1—Characteristics of Composite Organic-Inorganic Nanoparticles COINS. a) Transmission electron microscopy images of COINs, AOH (left) and BFU (right). b) Spectral signature of AOH COIN (red), BFU COIN (blue) and glass background (black) are indicated as Raman intensity. c) COIN aggregates enhance the Raman signal of SERS particles. The Raman intensity was measured for the dye alone (AO), silver (Ag) silver+dye (Ag+AO) and COIN-AOH and d) BFU [dye alone (BF), silver (Ag), silver+dye (Ag+BF), and (COIN-BFU) respectively. e) Raman intensity signal of COIN compared to the size of COIN cluster measured for AOH (red) and BFU (blue).

FIG. 2—Raman microscopy. a) Image of the “Integrated Raman BioAnalyzer”-IRBA. The arrow indicates the placement of the chamber with the sample prior to insertion into the apparatus. b) Generic configuration of Raman microscopic setup. c) Optimization of scans using IRBA. Wells containing COINs were scanned with a laser beam of 1 μm using a matrices of 5×5, 10×10, 15×15, 17×17 and 20×20 at 1 μm intervals at 100 μm distances. The spectra are indicated (left) and the calculated peak heights are represented as histograms (right). The experiments were performed 3 times in duplicates. The peak heights for the 15×15 and 17×17 are significantly different from the 5×5, 10×10 and 20×20; **p<0.01. d) Raman intensity of spectra from cells stained with different concentrations of αCD54-AOH-COIN (red—0.5 mM, blue—0.25 mM and yellow—0.1 mM) and AOH-COIN (purple—0.5 mM, green—0.25 mM and orange—0.1 mM), scanned by IRBA (left). Quantitation of the Raman peak height from the spectra observed illustrated as histograms *p<0.05 and **<0.01. The experiment was performed three times in duplicates.

FIG. 3—Specificity of COIN based Raman spectroscopy for detection of Surface antigens. a) Expression of ICAM (CD54) and absence of CD8 expression on U937 cells was determined by FLOW cytometry (left). Expression of CD54 on U937-expressing cells and H82-non-expressing cells was determined by FLOW cytometry (center). Expression of CD8 in a subset of human PBMCs was compared to non-expressing U937 and H82, determined by FLOW cytometry (right). b) Antigen specific detection of CD54 using COIN. Raman intensity of spectra from cells stained with αCD54-BFU and αCD8-BFU COINs (left). The spectra are representative for five independent experiments. Quantitation of Raman peak height is represented as histograms of five independent experiments performed in duplicates (right). The αCD54-BFU COINs specifically detected CD54 on U937 cells **p<0.01. c) Cell-specific detection of CD54 surface antigen using COIN. Raman spectra from CD54 expressing U937 cells and non-expressing H82 cells stained with αCD54-BFU COIN (left). Quantitation of Raman peak height is represented as histograms (right). The αCD54-BFU COINs specifically detected CD54 on U937 cells **p<0.01. d) SEM images of U937 cells stained with αCD54-BFU (left) and BFU (right) COINs. e) Characterization of a cell population in primary blood cells using COIN. Raman spectra of human PBMC, H82 and U937 cells stained with αCD8-BFU COIN (left). Quantitation of Raman peak height is represented as histograms (right). The αCD8-BFU COINs specifically detected CD8 on hPBMCs **p<0.01.

FIG. 4—Detection of intracellular phosphorylation signaling using COINs. a) Flow analysis of pStat1 and Stat6 phosphorylation following treatment of U937 cells with IFNγ or IL-4 cytokines, compared to non treated (Non stim). b) Raman spectral shift intensity of BFU COIN detecting intracellular pStat1 in IFNγ and c) pStat6 in IL-4, treated and non-treated (Non stim) U937 cells. Treated (Stim control) and non treated cells (Non stim control) were stained with non-conjugated BFU COIN. The spectra are representative for five independent experiments. d) Quantitation of change in Raman peak height after IFNγ and e) IL-4 treated cells compared to non-treated cells. The αpStat1 and αStat6 conjugated COINs specifically detected pStat1 and pStat6 respectively on IFNγ and IL-4 treated U937 cells compared to non-treated cells (**p<0.01). e) Fold change ratio of pStat1 and pStat6 phosphorylation in U937 treated cells stained with both BFU and AOH COINs. The changes are the average of five independent experiments.

FIG. 5—Detection of two intracellular phosphorylation events using two different COINs simultaneously. a) Raman spectra of the U937 cells treated with IFNγ and IL-4 simultaneously. The cells were stained with αpStat1-BFU and αpStat6-AOH simultaneously and separately. Cells were also stained with non-conjugated AOH and BFU COINs. The spectra are representative for five independent experiments. Cells were also stained with BFU and AOH that were not conjugated to antibodies. b) Extrapolated COIN spectra for treated (IFNγ+IL-4) and untreated cells (Non stim) stained with pStat1-BFU and BFU. c) Extrapolated COIN spectra for treated and untreated cells stained with pStat6-AOH and AOH. d) The Raman intensity of the Raman spectra for pStat1-BFU and pStat6-AOH COINs were calculated using the “MultiPle×” program (© Intel Corporation). The results are presented as histograms for single and double stain procedures. e) The fold change is the identified intensity of the spectra of the αpStat1 and αpStat6 conjugated COINs from treated and non-treated cells normalized to non-conjugated BFU and AOH COINs that were not conjugated to antibody. The results are the average of five independent experiments.

### DETAILED DESCRIPTION OF THE INVENTION

The ability to detect and identify trace quantities of analytes has become increasingly important in virtually every scientific discipline, ranging from part per billion analyses of environmental pollutants to analysis of biological samples. Raman spectroscopy is an analytical technique that provides rich optical-spectral information. It allows the detection and specific attribution of a signal among several simultaneously measured signals. A Raman spectrum, similar to an infrared spectrum, consists of a wavelength distribution of bands corresponding to molecular vibrations specific to the target analyte being analyzed. In practice, Raman spectroscopy employs a beam from a light source, generally a laser, that is focused on the sample to be analyzed in order to generate inelastically scattered radiation. That radiation is optically collected and directed into a wavelength-dispersive spectrometer in which a detector converts the energy of impinging photons to electrical signal intensity. Raman spectroscopy can exceed the limit of fluorescence emission overlap adjustment, as a fluorescent spectrum normally has a single peak with a half peak width of tens of nanometers (when using fluorescently labeled quantum dots) to hundreds of nanometers (fluorescent dyes). In contrast, a Raman spectrum typically has multiple bonding-structure-related peaks with half peak widths of as small as a few nanometers.

Spontaneous Raman scattering is typically very weak, and enhancement is required to improve the spatial resolution of the Raman scattering signal. Surface Enhanced Raman Scattering (SERS) techniques make it possible to amplify a Raman signal by 10³-10¹⁴ fold, and may even allow for single molecule detection sensitivity. Such enhancement is attributed primarily to enhanced electromagnetic fields on curved surfaces of coinage metals such as a copper, gold, and silver since their surface plasmons (containing valence electrons) are easily excited by laser light and produce an electric field that can be transferred to nearby Raman active molecules, i.e., “Raman labels”. By using a variety of Raman labels with distinct Raman spectral fingerprints, it is thus possible to generate a library of SERS molecules whose Raman spectra can be deconvoluted to determine the contribution of each individual signature in a combination of spectra. Thus, the nanoparticles can be used as a tool for multiple signal detection, which means that from 2-10, 10-50, 50-100, or even more than 100 target analytes can be simultaneously analyzed in a single experiment.

Although electromagnetic enhancement (EME) has been shown to be related to the roughness of metal surfaces or particle size when individual metal colloids are used, SERS is most effectively detected from aggregated colloids. Clusters of highly active nanoparticles SERS nanoparticles with highly enhanced Raman scatters have been created. See, for example, U.S. patent application publication numbers 20050147963, 20060068440, and 20050142567; Su, et al. (2005), Nano Lett., vol. 5: 49-54. These nanoparticles, termed “composite organic-inorganic nanoparticles” (COINs), are coalesced metallic nanoparticles with entrapped organic Raman labels. COIN clusters enhance the Raman signal by 10⁴⁻⁵ fold compared to single silver particles coated with Raman dye. This additional enhancement improves detection of Raman signal from COINs used in various biological and chemical assays, including immunoassays, and allows detection of biomolecules such as proteins nucleic acids in single cells comparable to fluorescence technology.

The COINs of the invention are preferably coated with protein such as albumin, including bovine serum albumin (BSA) or human serum albumin (HSA) to make them biocompatible (“bCOINs”). Protein encapsulation also unexpectedly facilitates bCOIN uptake into living (metabolically active) cells. COINs can be functionalized by association with (e.g., by covalent cross-linking, binding between the two members of a high-affinity binding pair (e.g., streptavidin and biotin) probe molecules such as antibodies, nucleic acids, receptors, receptor ligands, enzymatic substrates, second messengers, etc.

The invention will be better understood by reference to the following Examples, which are intended to merely illustrative and are not intended to be limiting in any way.

### EXAMPLES

The following examples describe the use of SERS-based COIN nanoparticles as analytical nanotags configured for immuno-detection in single cells, measuring epitopes on the surface of cells, as well as induced intracellular phospho-epitopes. The ability to deconvolute the Raman spectra of two simultaneous measurements of phosphorylation events in a single cell is also described. The signals detected by Raman spectroscopy are comparable to those measured by conventional flow methods. This study demonstrates the sensitivity of SERS-based COIN agents and their utility for analyzing biological events in single cells.

### Example 1

### SERS bCOIN Preparation

The SERS bCOIN clusters used in the experiments described in the examples were fabricated as silver nanoparticle aggregates initiated with either heat or salts in the presence of the organic Raman dyes Acridine Orange (AOH) and Basic Fuchsin (BFU). See Su, et al. (2005), Nano Lett., vol. 5: 49-54. Briefly, for AOH COIN fabrication, 12 nm silver seeds were prepared with silver nitrate (AgNO₃) and reduced by sodium borohydride (NaBH₄). The silver seeds were then mixed with sodium citrate (Na₃C₆H₅O₇) and 5-30 μM Acridine Orange Raman dye. The solution was heated at 95° C. for 60 min during which seed particles randomly grew with the adsorption of the Raman dye. The reaction was stopped by the addition of 0.5% Bovine Serum Albumin (BSA) (Roche, #10 238 040001). The BFU bCOINs were fabricated using Basic Fucshin as the Raman dye. The silver seeds were heated at 95° C. with 0.5 M AgNO₃ and Na₃C₆H₅O for 3 hrs. to enlarge the seeds to 24 nm. COIN clusters formed in the presence of 0.5 mM Basic Fucshin dye and 20 mM NaCl during a reaction time of 4 minutes. The process was stopped by the addition of 0.5% BSA.

The AOH and BFU COIN clusters were encapsulated with BSA to stabilize them and to introduce functional groups on the surface of the COINS to facilitate attaching various probe species, particularly various monoclonal antibodies specific for different biomolecular analytes.

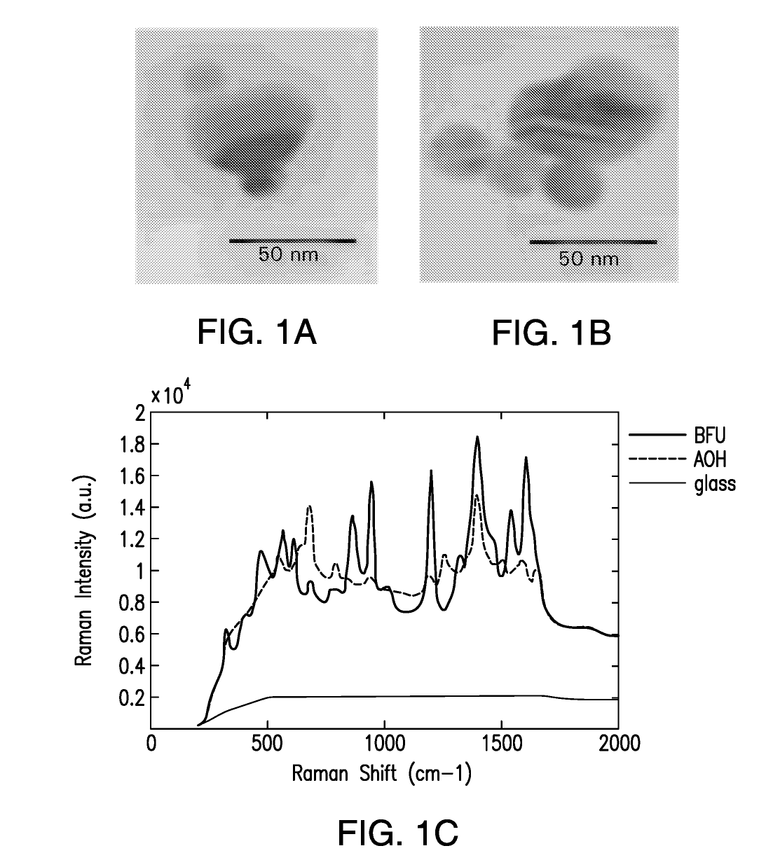

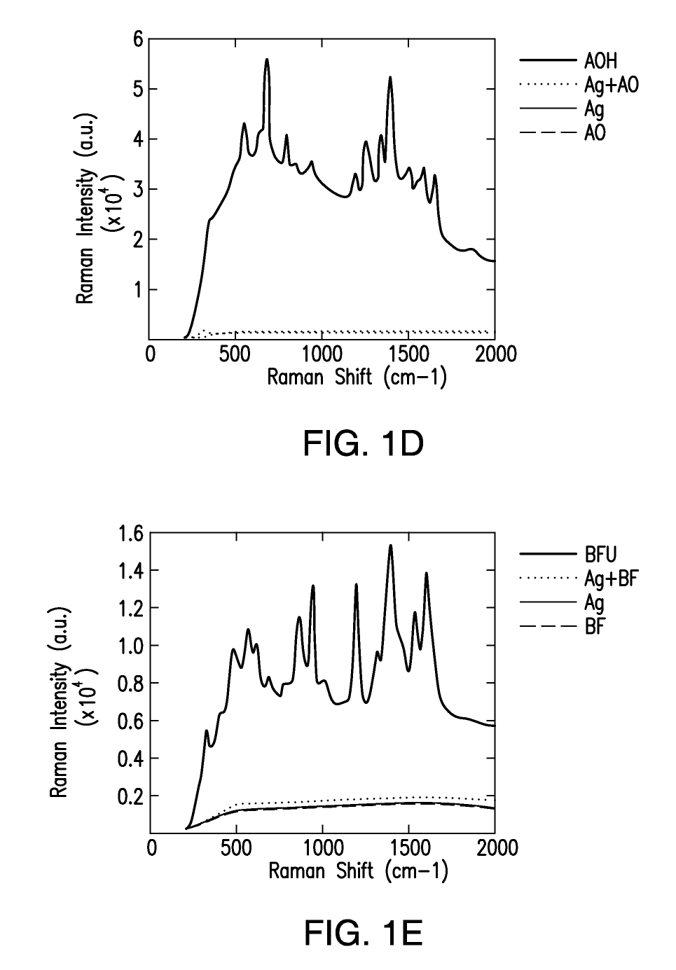

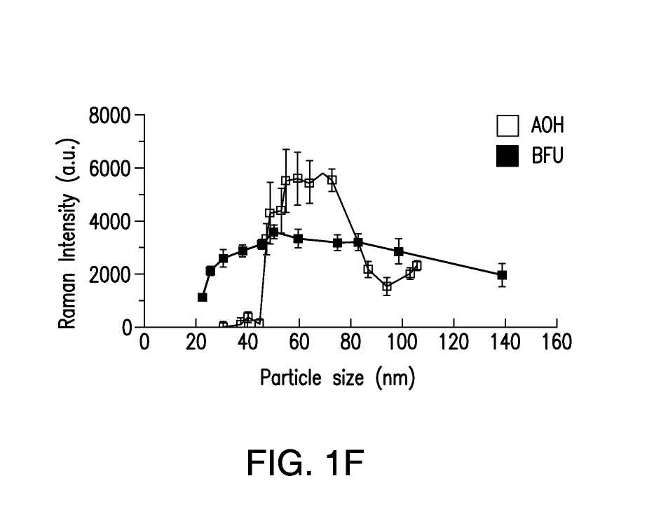

The AOH and BFU COIN clusters exhibited different Raman spectra. FIGS. 1A and B. The Raman intensity of spectra for COINs was significantly enhanced by the generation of clusters. Mixing of the silver seed particles with the Raman dye generated colloid silver particles with non-detectable Raman shifts. However, the aggregation of the silver particles into COIN clusters significantly enhanced the Raman signal intensity by approximately 10⁴-10⁵ fold. FIGS. 1C and D. To determine Raman activity related to COIN cluster size, COINs of increasing sizes were generated. The nanoparticle size and polydispersity was determined using photon correlation spectroscopy (PCS: Zetasizer, Malvern). The crude COINs were scanned for their Raman spectra using IRBA (see following paragraph). The intensity of the Raman spectra was found to increase with the size of the COIN particles. FIG. 1E. The trend was different for the different COINs. The Raman intensity for the AOH COINs increased abruptly when the mean size grew beyond 50 nm, and the intensity decreased when the particle size grew beyond 80 nm. The increase of the Raman signal for the BFU COIN was moderate but reached optimal intensity between 50-60 nm and decreased beyond that size. The COIN size suitable for bioassays was determined to be 60±6 nm for the AOH COIN and 52±5 nm for the BFU COIN, where the optimal intensity of the Raman peak was observed for each COIN. Thus, SERS-based COIN nanoparticles were generated that have specific and enhanced Raman shifts.

### Example 2

### Raman Microscopy

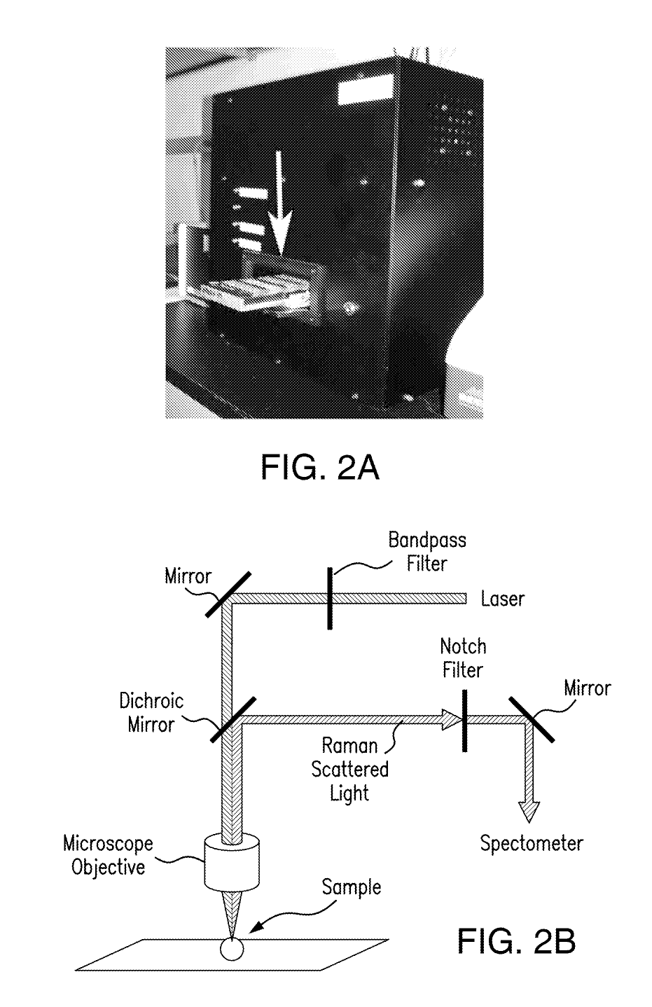

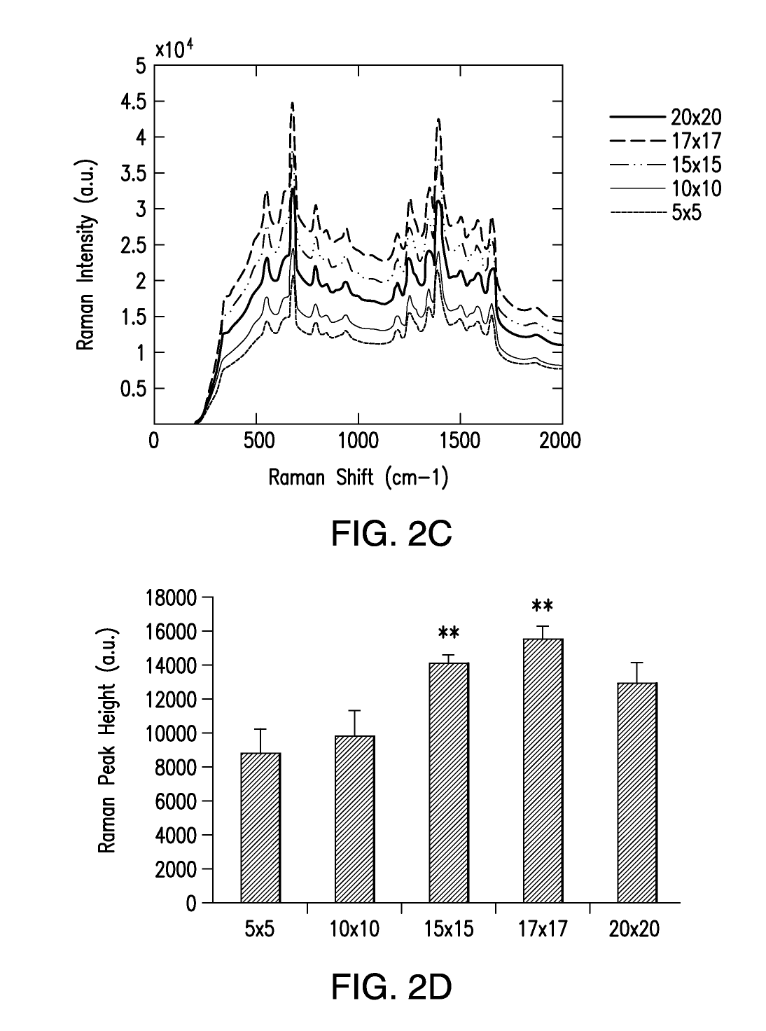

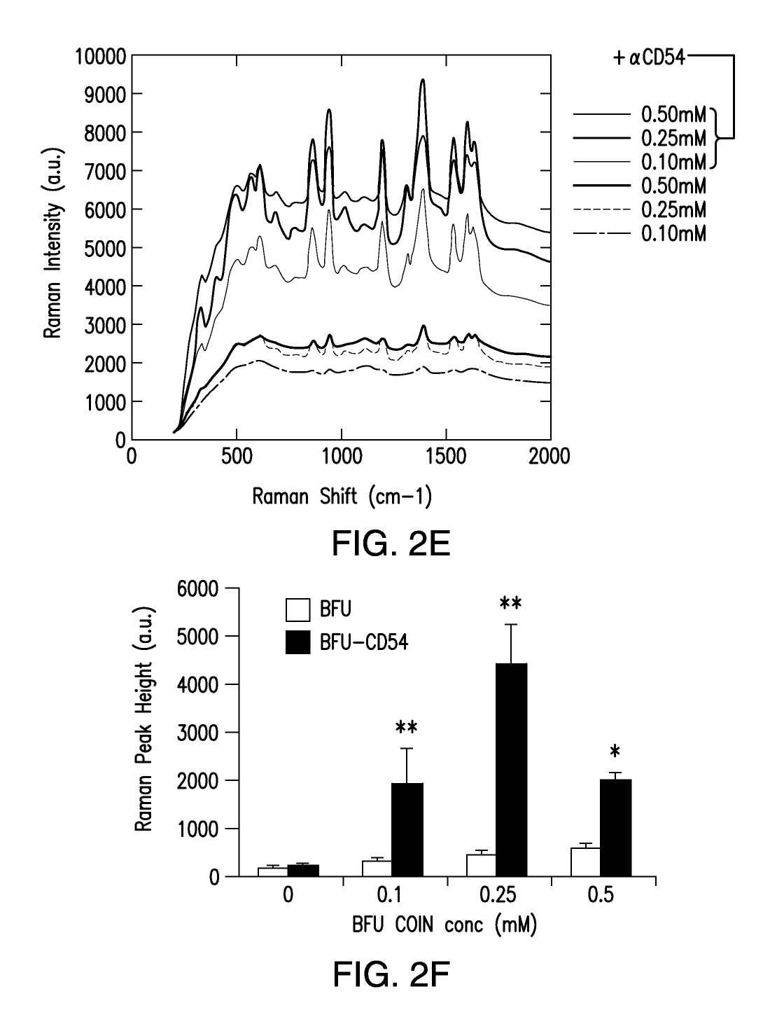

To reliably detect the Raman signal in a format appropriate for cellular analyses, an automated Raman scanner (Integrated Raman BioAnalyser—IRBA) was developed that is suitable for detecting Raman signals. A photograph of the device is shown in FIG. 2A. The schema for the IRBA is illustrated in FIG. 2B. The key components of the microscope are the dichroic filter and notch filter. The dichroic filter allows the laser light from a 532 nm excitation laser to reach the sample while reflecting all other wavelengths. The notch filter blocks the laser light while transmitting all other light wavelengths. The Raman scattering can be measured as spectral shifts as little as 30 nm from the excitation laser-light source, hence the slope of the notch filter is high (˜90 degrees).

The IRBA scans 64 wells in a microtiter plate-like format. Biological specimens were immobilized on aldehyde glass slides and assembled into a FAST Frame slide holder adopting the 64-well footprint. The sample wells were filled with phosphate buffered saline (PBS), covered with cover glass and loaded into the sample tray holder of the IRBA (see arrow in FIG. 2A). During a scan, samples were probed by a continuous wave, diode-pumped, solid-state laser. The IRBA is prompted to automatically focus the laser beam onto the sample using an aspheric objective lens with a f/0.5 numerical aperture and a 20× magnification. The laser power at the sample stage is 100 mW, with a laser spot size ˜1 μm in diameter. A mechanical shutter reduces the sample exposure to laser light. A typical exposure time is 0.1 seconds per spot. The detector is a back-illuminated, thermoelectrically-cooled CCD camera. The IRBA conducts automated data acquisition from the slide using a user-defined raster scan. The IRBA configuration was set up to collect a single Raman spectrum from a 1 micron spot at a distance of 10 microns with an acquisition time of 100 ms. The IRBA performed a raster scan of the sample-containing wells using a scan matrices of 2×2 up to 20×20 with 100 μm intervals. The optimal raster scan was tested using an AOH COIN solution. An increase in the Raman intensity signal was found with the increase in scan parameters. The optimal results were obtained using a scan matrix of 17×17 matrices with 100 μm intervals (FIG. 2C). Thus, the Raman scanner could scan a sample plated in a well-chamber, making it suitable for further analysis of cells, as detailed below.

### Example 3

### Detection of Cell Surface Antigens

This example describes the testing of the AOH and BFU COINs in immunoassays. First, the ability to use COIN nanoparticles to detect surface antigens on single cells stained in suspension was assessed. Antibodies were conjugated to COINs. Antibodies were conjugated to the BSA encapsulated COINs. See Sun, et al. (2007), Nano Lett, vol. 7: 351-6. The carboxylic groups on BSA were activated with N-(3-(dimethylamino)-propyl)-N/-ethylcarbodiimide (EDC) (Sigma, #39391). Antibodies used for COIN conjugation were: CD54 (BD, #550302), CD8 (BD Bioscience, 554716), pStat1 (Y701) (BD Biosciences, #612596), and pStat6 (Y641) (BD Biosciences, #612600).

The ability of an antibody-conjugated COIN to function in a bioassay was initially determined in an IL-8 ELISA sandwich assay. Aldehyde treated slides (NUNCT″, #23164) were coated with IL8 capture antibody (BD Pharmingen, #554716) mounted on FAST® frames (Whatman Inc., #10486 001). We added 1-100 ng of IL8 was added to the wells for 15 minutes and then washed with PBST (×2). BFU or AOH COINs conjugated to αIL8 antibody (BD Pharmingen, #554717) were used to stain the wells for 1 hour at room temperature (RT). The wells were washed in PBST (PBS and 0.1% Tween 20) and 0.1 M NaCl. The wells were filled with PBS and covered with cover glass (VWR International, #48366 067). The Raman spectra was measured for each well using the IRBA running a 532 nm excitation laser. The COINs that passed the initial quality control criteria were used for further detection assays. The criteria were: 1) experimentally-derived linear relationship between IL-8 concentration and Raman intensity readings (r²=0.8-1); and 2) the COIN should not precipitate during antibody conjugation. The IL-8 antibody-COIN conjugate that showed a linear reactivity to IL-8 antigen concentration with a linear slope (r²>0.8) was considered suitable for further. Both the AOH and BFU COINs, representing two different fabrication processes, passed the initial control and were considered suitable for use in other biological assays.

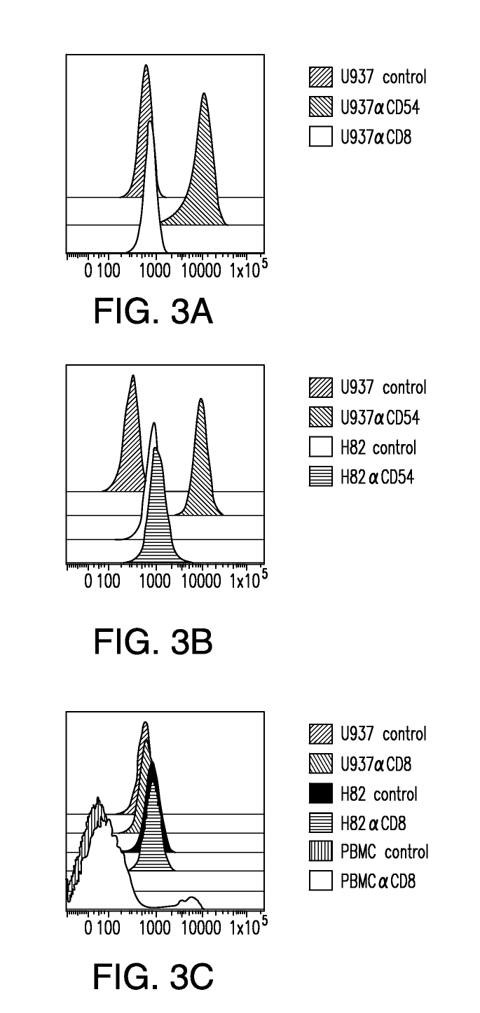

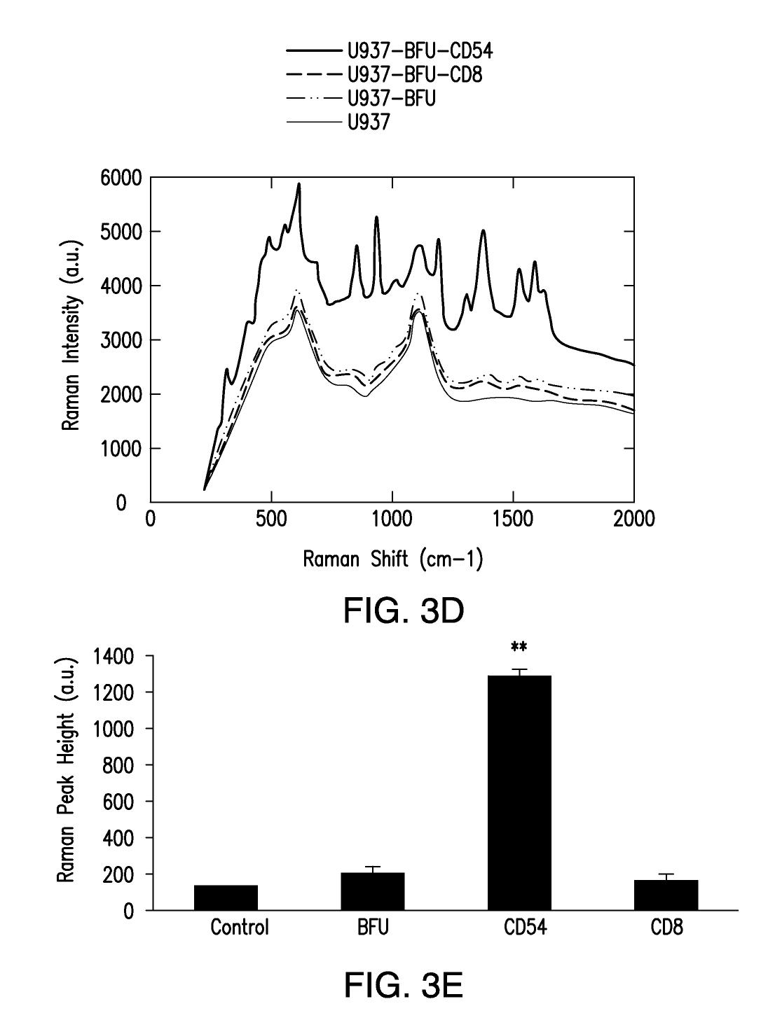

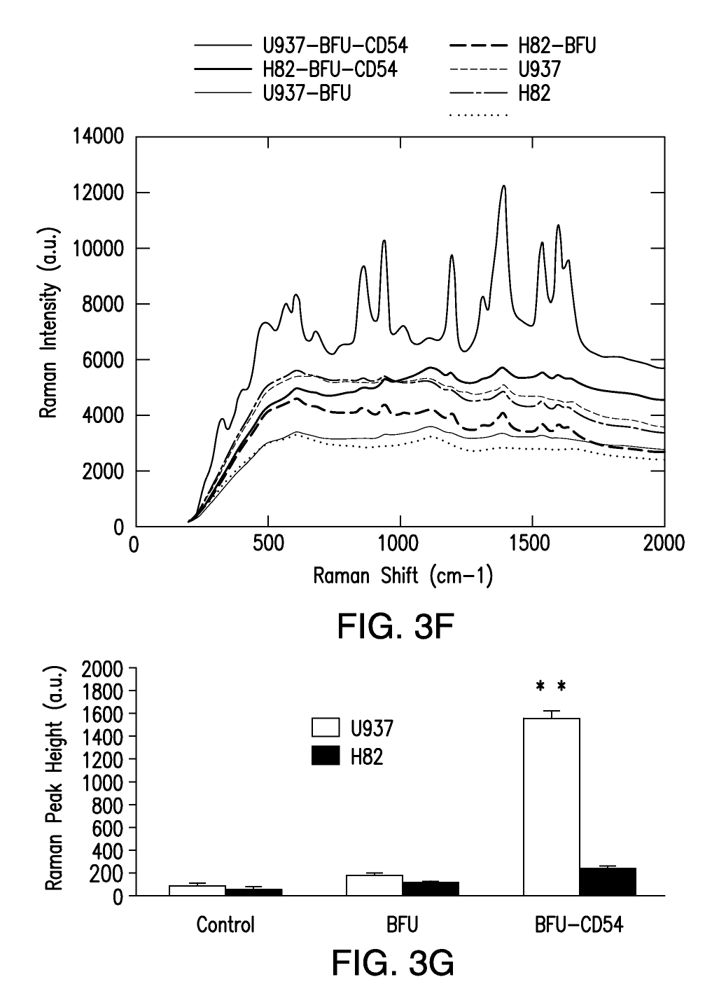

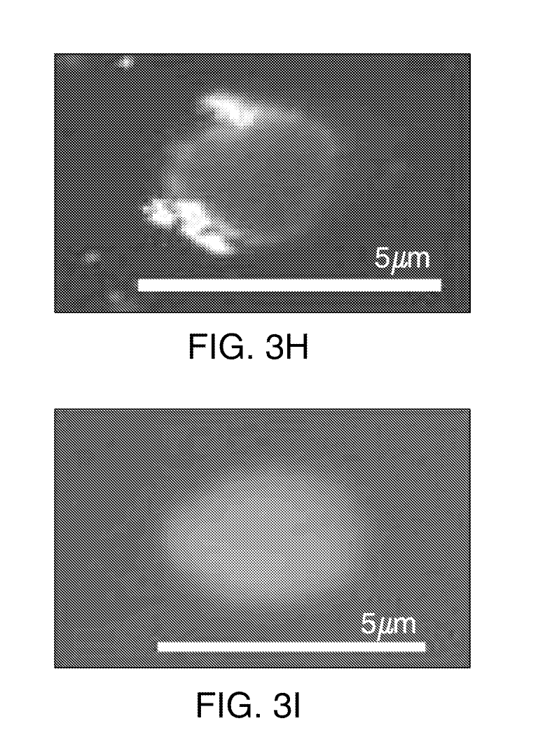

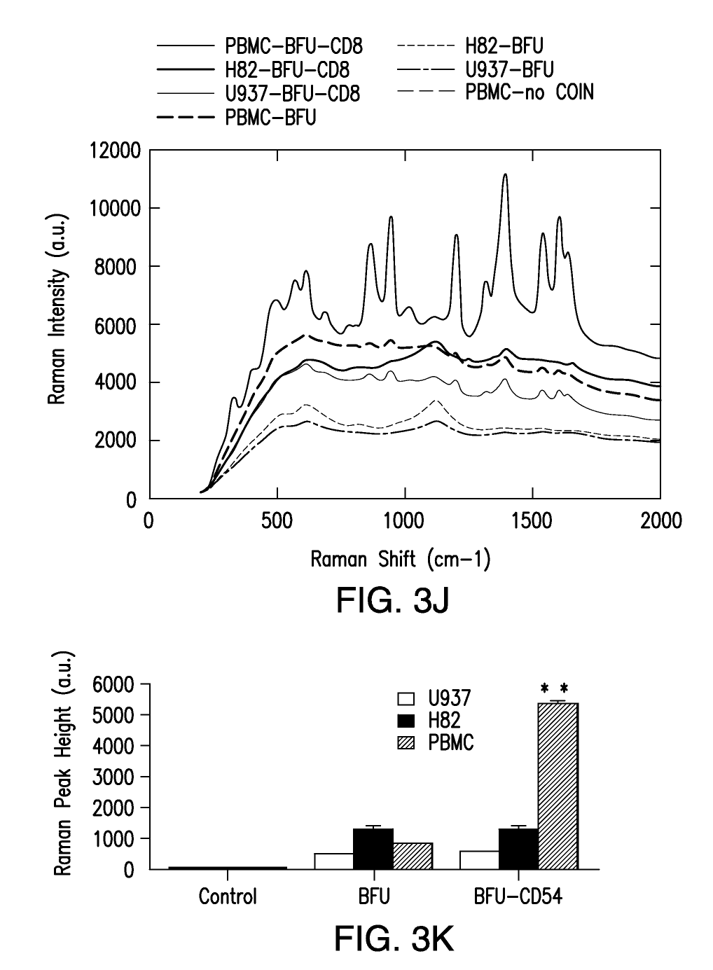

To further determine the utility of the COINs as detectors, measurements of surface proteins expressed in the U937 cell line were performed. The U937 cell line is a monocytic leukemia with high ICAM-1 (CD-54 adhesion molecule) expression on the cell surface (FIG. 3A, left & center). The AOH and BFU COINs were conjugated with anti-CD54 antibodies and used to detect the CD54 antigen in an ELISA. Linear regression analysis of COIN signal versus antigen concentration in the ELISA yielded correlation coefficients (r²) of 0.8-0.99. A CD54-COIN ELISA direct-binding assay was performed as described above using monoclonal αCD-54 antibody (BD Pharmingen, #555364) conjugated to AOH or BFU COINs. Wells were coated with 5 ng/ml-500 ng/ml recombinant human CD-54 (1-CAM-1) protein (R&D, #ADP4-200). An experimentally-derived linear relationship between CD54 protein concentration and αCD54-COIN Raman intensity readings (r²=0.8-1) was used to determine that the COINs passed the initial quality control studies. These antibody-COIN conjugates were used for further cell staining procedures. Thus, both the AOH and BFU COINs were found suitable to be used in cell-staining to analyze the CD 54 antigen on the cell surface.

The optimal concentration of the COIN in the surface staining protocol was determined by Increasing concentrations (0.1, 0.25, 0.5 mM) of COIN+αCD54 incubated with U-937 cells. Excess unbound COIN was washed off and 0.5×10⁶ cells were spun down in the scanning chamber wells. The chamber-containing cells were scanned using IRBA and the 17×17 scan protocol, previously determined as optimal. The average spectrum was calculated for the spectra acquired for each well (FIG. 2D). Raman peaks for the COIN signal were defined, and peak heights were calculated. The peak heights are displayed as histograms in FIG. 2D. An increase in BFU-COIN specific peak height was found with an increase in concentration from 0.1 to 0.25 mM, and a decrease in peak height was observed when COIN concentration increased to 0.5 mM. A similar trend was observed for the AOH COIN. The optimal concentration for COIN staining for further experiments was determined to be 0.25 mM.

To assess the accuracy of COINs for detecting specific surface antigens, the ability of the COINs to bind to CD54 antigen expressed on U937 cells was compared to CD8 antigen that is not expressed on U937 cells. FIG. 3A, left panel. The spectra for cells stained with antibody-conjugated COIN and non-conjugated COIN was obtained (FIG. 3B, left). The peak heights for each spectrum were quantitated and are represented as histograms (FIG. 3B, right). The Raman peak ratios were determined for the relative Raman peak heights of antibody-conjugated COIN compared to non-conjugated COIN. A specific reactivity of the αCD54-antibody conjugated COIN in U937 cells was obtained. Both the AOH and BFU COINs showed similar detection reactivity to CD54 on the surface of U937 cells. To determine the cell-specific binding of the COINs, H82 small cell lung cancer (SCLC) cells that do not express CD54 (FIG. 3A, middle panel) were also stained. Specific binding of the αCD54-COIN to CD54 expressing U937 cells but not to H82 cells was observed. FIG. 3C. The results using the BFU COIN were comparable to the AOH COIN.

To visualize the localization of CD54-COIN on the cell surface, U937 cells were analyzed by Scanning Electron Microscopy (SEM). The cells stained with COIN without additional processing were imaged, which is usually required for SEM, by using Quantomix capsules. Using SEM on native samples, clusters of COINs we detected at the apex of U937 cells (FIG. 3D), which is characteristic for the expression of CD54.

To determine the ability of COINs to stain primary human cells, human peripheral blood mononuclear cells (PBMC) were stained with αCD8-conjugated COINs. A subset (˜7%) of the total hPBMCs was CD8+ T-cells, as measured by flow cytometry. FIG. 3A, right. A αCD8-COIN signal was detected in PBMC but not in either U937 or H82 cells. FIG. 3E. To determine if only a subset of the cells reacted to the αCD8-conjugated COIN, each scan for Raman spectra was examined. Approximately 10% of the scans yielded Raman spectra correlating with specific COIN signals. This percentage of positive signals compares to the range of cells positive by FLOW cytometry. The stain was repeated, now using AOH COIN. The results observed with the BFU COIN were comparable to those obtained using the AOH COIN. Peak heights were determined using the PeakHeight software (© Intel Corporation) in MATLAB (The MathWorks, Inc). Peak height areas were calculated using the following parameters: peak-start, peak-top and peak-end for each spectrum.

These results lead to the conclusion that antibody-conjugated COINs bind specifically to antigens when used for immunostaining of single cells. While the intensity of the Raman peak height may vary for each COIN, the calculated Raman peak height ratio of the antibody-conjugated COIN compared to non-conjugated COIN was similar for both AOH and BFU.

To conduct the experiments described in this example, U937 cells (ATCC-CRL-1593.2) were cultured in RPMI medium (Invitrogen, Carlsbad, Calif.). hPBMCs were isolated using density gradient solution (Ficoll-Paque Plus; Amersham Biosciences). Cells were washed in PBS and fixed in 1.5% paraformaldehyde (Electron Microscopy Sciences, Hatfield) for 15 minutes. The cells were washed in PBS then blocked with 1% BSA (Fraction V (Sigma, #A4503) for 1 hour during rotation. The cells were then washed in PBS (×1) and COIN staining buffer (×1) (PBST (PBS and 0.1% Tween 20)+10% fetal bovine serum (HyClone). 2×10⁶ cells were stained in 200 μl COIN staining buffer with 0.1, 0.25, and 0.5 mM concentration of COINs. The stained cells were then washed with PBST (×2) and then with PBS (×1) to remove the detergent. 0.5×10⁶ cells were immobilized by centrifugation at 1800 g for 15 min on 0.5% gelatin coated aldehyde slides (G7765, Sigma) fixed on FAST® frames (Whatman Inc., #10486 001). The supernatant was removed from the wells and replaced with 200 μl PBS. The wells were then covered with cover glass (VWR International, #48366 067). The Raman spectra were measured using the IRBA and a 532 nm excitation laser.

### Example 4

### Detection of Intracellular Phosphorylation

This example describes testing the potential of COIN nanoparticles to detect intracellular phosphorylation events. U937 cells activate intracellular signal transduction pathways when treated with IL-4 (Peprotech, #300-02) and IFNγ (Peprotech, #200-04). For treatment, U937 cells were suspended in RPMI media at the concentration of 5×10⁶ cells/ml. The cells were treated for 15 minutes at 37° C. with 20 ng/ml of human IFNγ (Peprotech, #200-04) to induce Stat1 phosphorylation or 20 ng/ml of human IL-4 (Peprotech, #300-02) to induce pStat6 phosphorylation. Cells were fixed in 1.5% PFA for 15 minutes, washed in PBS, suspended in 70% ethanol, and stored at −80° C. Before staining with COIN, the cells were washed in PBS and fixed in 1.5% PFA for 15 minutes at RT. The same staining protocol described above was used for the detection of surface proteins.

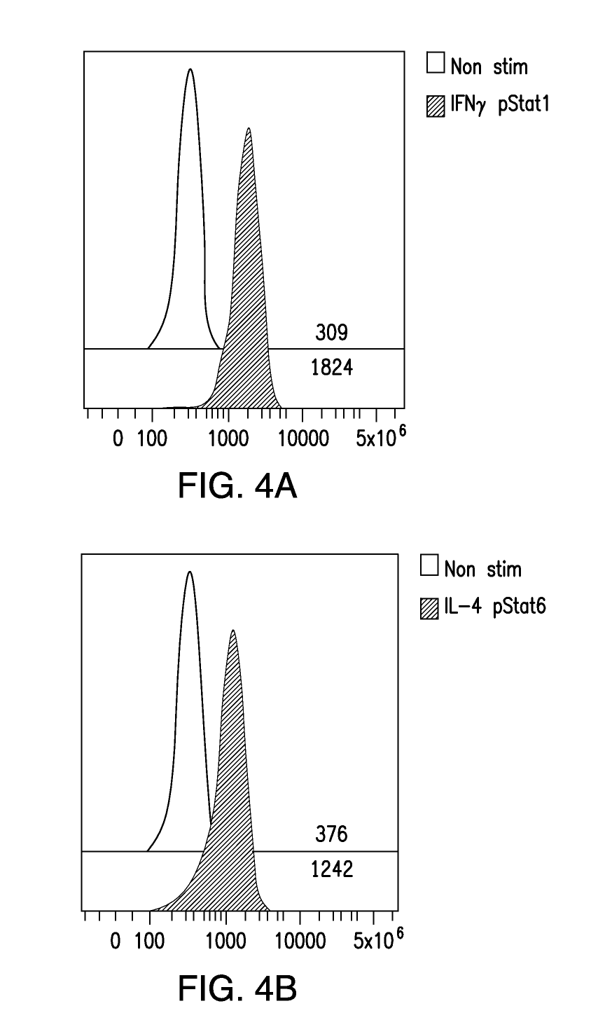

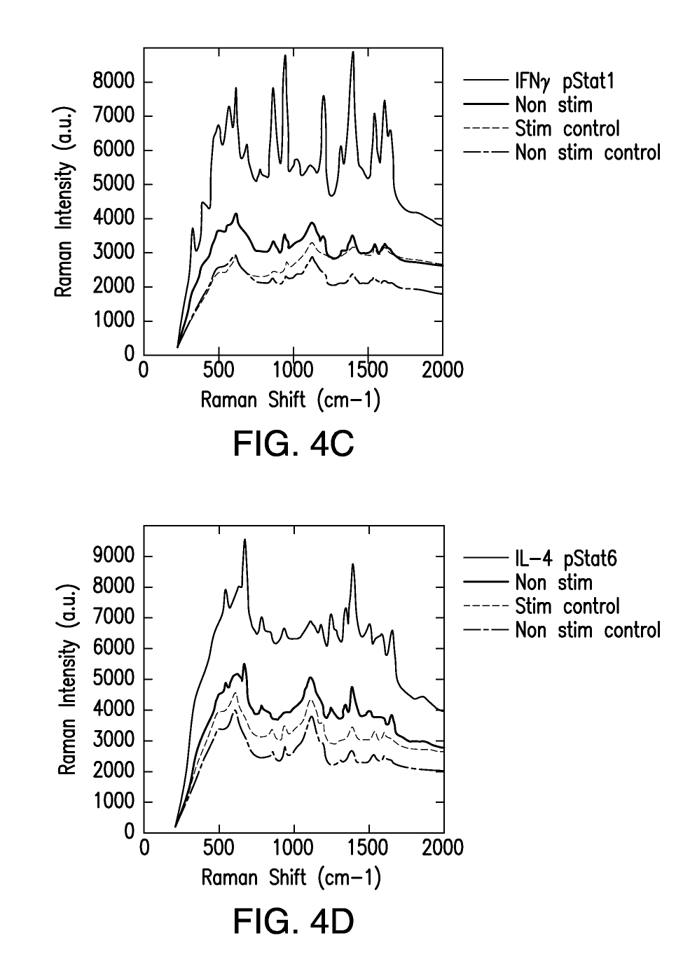

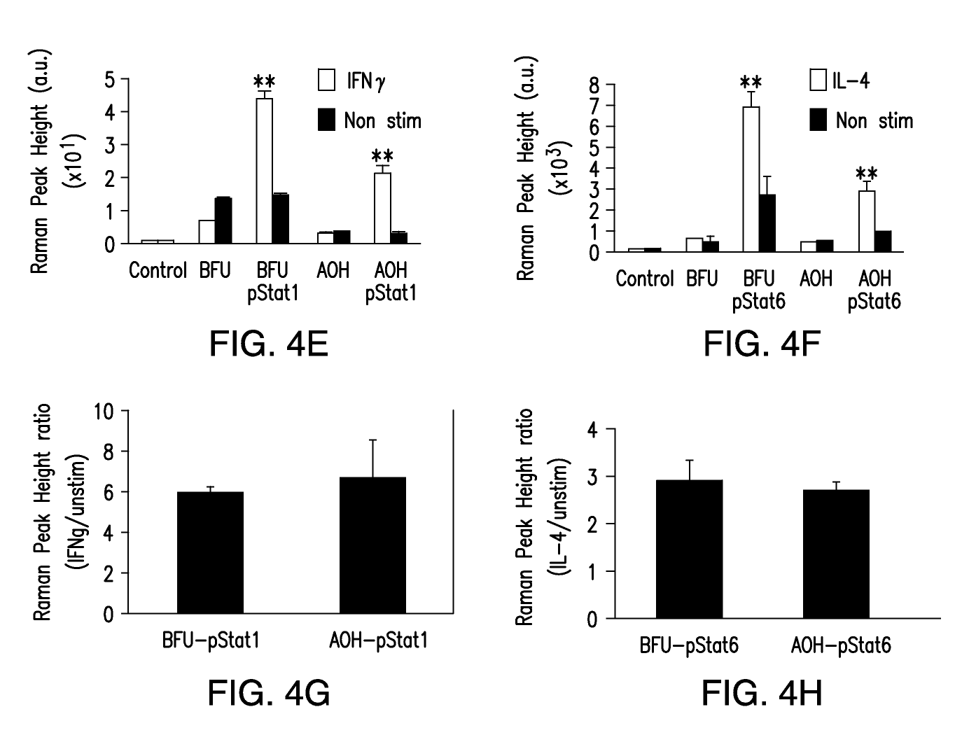

Treatment of U937 cells with IL-4 induces the phosphorylation of Stat6, while treatment with IFNγ induces the phosphorylation of Stat1. The increase in phosphorylation of Stat1 and Stat6 was first confirmed by PhosphoFlow analysis. FIG. 4A. A 5.9 fold increase of the phosphorylation of pStat1 was measured following IFNγ treatment and 3.3 fold increase in phosphorylation of pStat6 following IL-4 treatment. BFU and AOH COINs were conjugated to antibodies that recognize the Y701 phosphorylated epitope of the Stat1, and the Y641 epitope of the Stat6 proteins. The cells were then fixed and permeabilized. See Krutzik and Nolan GP (2003), Cytometry A, vol. 55: 61-70.

A pStat1 COIN sandwich assay was also performed. Rabbit monoclonal aStat-1 antibody (Cell Signaling Technologies, #9175) was used as the capture antibody. 0-10 μg pStat1 blocking peptide (Cell Signaling Technologies, #1038) was incubated in the antibody-coated wells. The pStat1 (pY701) mouse monoclonal antibody (BD BioScience, #612233,) was purified using Protein G and Protein A orientation kits (PIERCE, #44990), then conjugated to the AOH or BFU COINs. An experimentally-derived linear relationship between pStat1 peptide concentration and αpStat1-COIN Raman intensity readings (r²=0.8-1) was used to determine that the COINs passed initial quality control. These antibody-COIN conjugates were used for further cell staining procedures.

To prevent non-specific binding of COIN to intracellular proteins, an additional fixation step was carried out. Non-treated and treated cells were stained with antibody-conjugated and non-conjugated COIN washed and scanned using IRBA. The average spectra for IFNγ and IL-4 treated and non-treated cells are shown for AOH-pStat6 (FIG. 4D) and BFU-pStat1 (FIG. 4C). To determine if the COIN itself affects the binding ability, the antibodies were alternated on each COIN. The changes in peak height were determined and the ratio of the Raman signal in treated cells was compared to non-treated cells. FIG. 4C. A 5.9 fold change was detected in pStat1 phosphorylation using αpStat1-BFU COIN and a 6.7 fold change using αpStat1-AOH COIN. A 2.9 fold change was measured in pStat6 phosphorylation using αpStat6-BFU COIN and a 2.7 fold change in using αpStat6-AOH COIN. The detected changes in phosphorylation of the Stat1 and Stat6 molecules using the AOH or the BFU COINs was similar to what was observed by PhosphoFlow.

These results demonstrate the utility of COINs for measuring intracellular phosphorylation events in single cells.

### Example 5

### Simultaneous Detection of Multiple Raman Signals

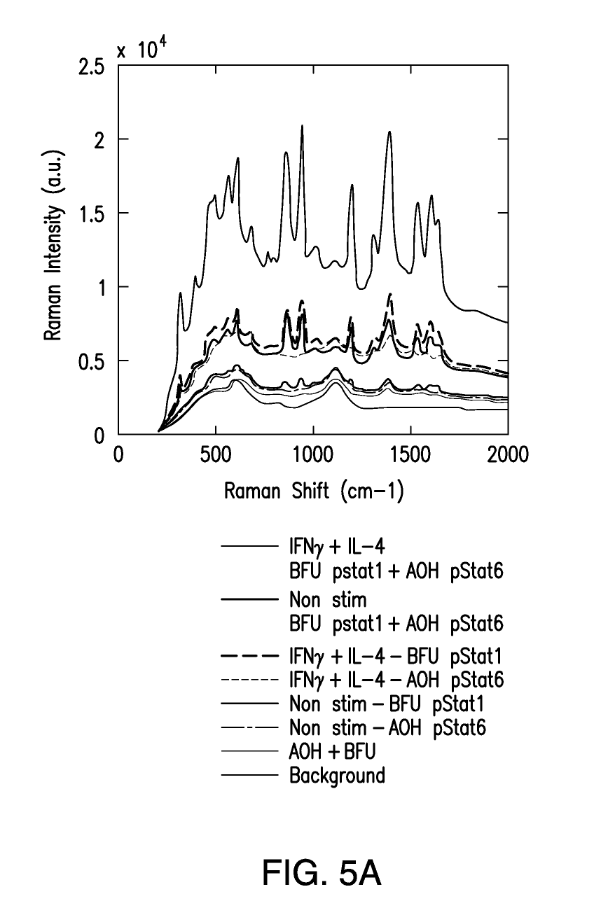

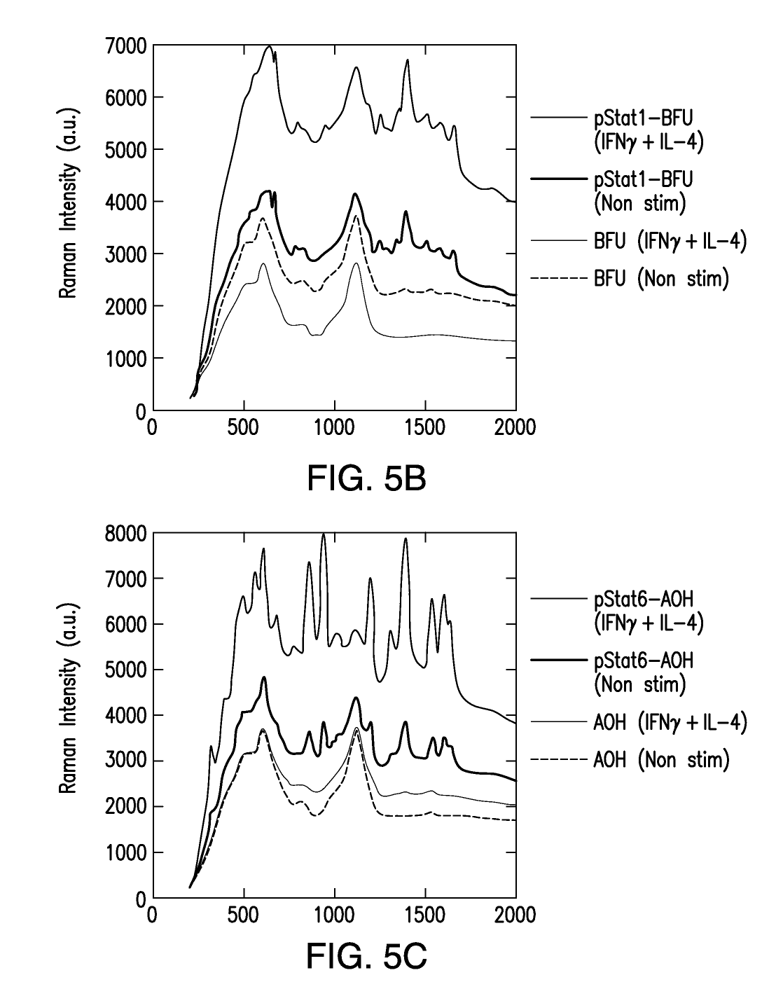

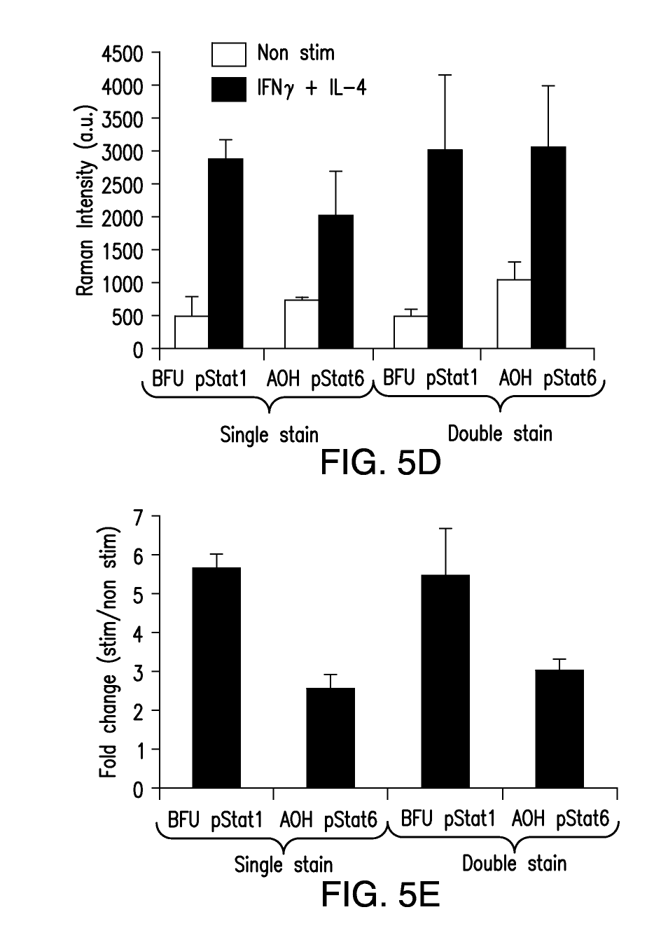

This example describes the conduct of representative intracellular multiplex assays using COINs. A multi-parameter analysis was designed and simultaneous stained cells with AOH and BFU COINs, for detecting two phosphorylation events in a single cell. U937 cells were co-treated with IFNγ and IL-4. Simultaneous staining of the cells was conducted using BFU conjugated to pStat1 and AOH conjugated to pStat6 antibody. Cells were also stained with BFU-pStat1, AOH-pStat6, and non-conjugated BFU and AOH COINs as controls. The cells were then scanned using the IRBA running a 532 nm excitation laser and the Raman signal intensities detected from the samples are displayed. FIG. 5A. The “MultiPle×” program (© Intel Corporation) run in MATLAB (The MathWorks, Inc.) was used to deconvolute the two Raman spectra detected simultaneously from the BFU and AOH COINs. The representative Raman spectra for each COIN was identified and deconvoluted using the “Least Squares Method” to determine the spectral contribution from the different sources. Spectra for untreated cells was then extracted, treated cells for the pStat1-BFU and pStat6-AOH COINs. FIGS. 5 B and C. Peak heights representative for each spectrum were measured and the changes in ratio of the antibody-conjugated COIN peaks were compared to non-conjugated COIN, in treated and non-treated cells. FIG. 5D. The results from the double assay were also compared to the single assay in the experimental setup. A 5.4-fold increase in pStat1 was measured in the double stain compared to a 5.7-fold change in the single stain experiment. A 3.1-fold increase was detected in pStat6 in the double stain compared to a 2.9-fold change in the single stain experiment. The calculated changes in peak height ratio were statistically similar when two COINs were used simultaneously compared to using a single COIN in a staining assay.

To illustrate the robustness of simultaneous staining procedures for phospho-epitopes with COINs, cells were treated with IFNγ (pStat1) or IL-4 (pStat6) or IFNγ/IL-4 (pStat1/pStat6). The cell samples were stained simultaneously with both COINs; the BFU COIN conjugated to pStat1 and the AOH COIN conjugated to pStat6 antibody. The samples were scanned using the IRBA. The Raman spectra were deconvoluted using the MultiPlex program (© Intel Corporation). The Raman peak heights were calculated and represented as histograms. The peak heights from cells stained with antibody conjugated COINs were normalized to the Raman signal from cells stained with non-conjugated COINs. The Raman signal from cells treated with either IFNγ or IL-4 cytokine was statistically similar to the signal from cells stained with both cytokines simultaneously (p>0.2).

These data demonstrate the use if COINs for the measurement of two simultaneous phosphorylation events in a cell staining assay.

These studies demonstrate the ability to use SERS bCOIN nanoparticles for multi-plex immuno-detection in single cells. Multiple and distinct COIN Raman nanoparticles can be generated with resolvable signatures that can be used to detect surface antigens and to measure changes in intracellular analytes and processes, including phosphorylation events. Enhanced Raman signatures via SERS and COIN technology offers capabilities that exceed fluorescent dye technology limits. COIN Raman spectra have several sharp peaks that define a “fingerprint” for each COIN. Multiple COIN spectra can be readily collected and deconvoluted. The detection of Raman signal of COINs, whose Raman detection is independent of fluorescence, provides a dramatic increase in the multiplicity of simultaneous measurements that can be taken in a single assay or experiment. Another advantage of Raman COIN technology is its versatility. The Raman spectra of COINs are measured as a shift relative to the excitation wavelength. The excitation of fluorophores is confined to a specific wavelength and re-emits energy at different (but very specific) wavelengths. COINs, on the other hand, can be excited by different wavelengths depending on the available equipment.

In conclusion, Raman COIN technology is a powerful tool that will be useful for multi-parameter simultaneous measurements of events inside even single cells. By enhancing the capacity to measure intracellular events at the single cell level, studies of cellular processes are possible. Thus, studies that use, for example, intracellular potentiation as a marker of biochemical processes, clinical outcome in primary patient materials, or for determinations of signaling networks by computational processes, can be performed.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, published patent applications, and other publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method for intracellular analyte analysis, comprising:
a. contacting a population of cells known or suspected to contain or present a target analyte (TA) species with an analytical nanotag (ANT) species comprising a TA species-specific probe bound to a biocompatible composite organic-inorganic nanoparticle (bCOIN); and
b. analyzing the cells to assess intracellular formation of TA:ANT complexes, thereby conducting an intracellular analyte analysis.

2. A method according to claim 1 wherein the cells are living [metabolically active] cells.

3. A method according to claim 1 wherein the target analyte is selected from the group consisting of a protein, a peptide, a small molecule, and a nucleic acid.

4. A method according to claim 1 wherein the analytical nanotag (ANT) species comprises a biocompatible COIN species comprised of a metal species, optionally copper, gold, palladium, platinum, or silver, and an entrapped organic Raman label species.

5. A method according to claim 1 wherein the analytical nanotag (ANT) species comprises clustered COIN species comprised of a metal, optionally copper, gold, palladium, platinum, or silver, and an entrapped organic Raman label species, wherein the clustered COIN species is coated with a biocompatible layer conjugated to the TA species-specific probe.

6. A method according to claim 5 wherein the TA species-specific probe is selected from the group consisting of an antibody or antibody fragment, a receptor or receptor fragment, and a probe nucleic acid.

7. A method according to claim 5 wherein the biocompatible COIN comprises a protein-encapsulated COIN, wherein the protein encapsulating the COIN optionally is an albumin, optionally bovine or human serum albumin.

8. A method according to claim 1 configured for simultaneous analysis of a plurality of TA species, wherein the population of cells is contacted with a plurality of ANT species each comprising a different TA species-specific probe species and different biocompatible COIN species.

9. A method according to claim 1 wherein the analysis of cells comprises Raman spectroscopy.
